(19) United States
(12) Patent Application Publication
(10) Pub. No.: US 2006/0151325 A1
(43) Pub. Date: Jul. 13, 2006

(54) Method for ph-biased isoelectric trapping separations

(76) Inventor: Gyula Vigh, Magnolia, TX (US)

Correspondence Address: COOLEY GODWARD LLP, THE BROWN BUILDING - 875 15TH STREET, NW, SUITE 800, WASHINGTON, DC, 20005-2221, US

(21) Appl. No.: 10/516,734

(22) PCT Filed: Jun. 3, 2003
(86) PCT No.: PCT/US03/17300

§ 371 Date: Feb. 6, 2006

(30) Foreign Application Priority Data
Jun. 5, 2002 (US) 10/163885

(51) Int. Cl. C07K 1/26; B01D 57/02

(52) U.S. Cl. 204/548; 204/450; 204/543

(57) ABSTRACT

A method for separating an ampholytic component by electrophoresis, the method involving placing a sample containing an ampholytic component having a pI value in an electrophoresis separation system comprising an anolyte having a pH and a catholyte having a pH, the catholyte pH being higher than the anolyte pH, one or more ion-permeable barriers disposed between the anolyte and catholyte wherein at least one of the barriers is an isoelectric barrier having a pI value which is higher than the anolyte pH and lower than the catholyte pH; providing an isoelectric buffer having a pI value higher than the pH of the anolyte and lower than the pH of the catholyte and different from the pI value of the ampholytic sample component and different from the pI value of an ion-permeable isoelectric barrier; and exposing the sample to an electric potential so as to trap the ampholytic sample component in a non-isoelectric state in the presence of the isoelectric buffer in the electrophoresis system.

### TECHNICAL FIELD

The field of the present application is the separation and concentration of ampholytic compounds. More specifically, the present application relates to a method for altering the composition of samples that contain at least one ampholytic sample component employing pH-biased isoelectric trapping techniques.

### BACKGROUND

Electrophoretic techniques, and isoelectric focusing (IEF) techniques in particular, remain key technologies for the separation of ampholytic components, small and large, simple and complex alike. Used in many fields and industries, IEF is performed both on an analytical and preparative scale. For example, IEF is utilized in clinical diagnosis, biotechnology, pharmaceutical and food industries, etc., alone or coupled with other analytical or preparative techniques.

In IEF, ampholytic components are separated with the help of an electric field in a pH gradient wherein the pH increases from a lower pH value at the anode to a higher pH value at the cathode. (For a monograph on IEF, see, e.g., P. G. Righetti, Isoelectric focusing: theory, methodology and applications, Elsevier Biomedical, Amsterdam, 1983, which is herein incorporated by reference.). Since the net charge of an ampholytic component is zero in its isoelectric state, the electrophoretic migration velocity of an ampholytic component becomes zero whenever the pH of its environment becomes equal to its isoelectric point (pI) value. Thus, ampholytic components with different pI values stop migrating at different points in the pH gradient.

Relatively stable continuous pH gradients can be created by several means. For example, mixtures of carrier ampholytes (compounds that have adequate buffering ability and conductivity in the vicinity of their pI value) may be used. Also, appropriate amounts of suitable weak acids and weak bases or weak acids and strong bases or strong acids and weak bases may be bound, in a spatially controlled manner, into an ion-permeable matrix, such as a cross-linked polyacrylamide gel to preform and stabilize the pH gradient which is then used for immobilized pH gradient IEF (IPGIEF). (For a monograph on IPGIEF, see, e.g., P. G. Righetti, Immobilized pH gradients: theory and methodology, Elsevier, Amsterdam, 1990, which is herein incorporated by reference.).

Alternatively, ampholytic sample components can also be separated from each other by isoelectric trapping (IET) utilizing isoelectric membrane-based multicompartmental electrolyzers (e.g., Faupel et al., U.S. Pat. No. 5,082,548, which is herein incorporated by reference) wherein at the end of an IET separation process, ampholytic sample components are obtained in their isoelectric state.

### SUMMARY OF INVENTION

Embodiments of the present application electrophoretically alter the initial composition of a sample that contains at least one ampholytic component using a pH-biased isoelectric trapping (IET) process by adding one or more suitable isoelectric buffers that have adequate buffering capacity, titrating capacity, and conductivity in the vicinity of their pI values to obtain an ampholytic sample component in its non-isoelectric state at the end of the IET process.

Briefly, a method practiced according to the present application provides for selecting an electrophoretic separation system having at least one ion-permeable isoelectric barrier, selecting an anolyte having a pH. value lower than the pI value of the ampholytic sample component, selecting a catholyte having a pH value higher than the pI value of the ampholytic sample component, providing an isoelectric buffer having a pI value higher than the pH value of the anolyte and lower than the pH value of the catholyte and different from the pI value of the ampholytic sample component, introducing the sample and the isoelectric buffer into the electrophoresis system and trapping the ampholytic sample component in a non-isoelectric state by executing an electrophoretic separation.

One aspect provides for selecting an electrophoretic separation system having one ore more separation compartments. Another aspect provides for selecting two or more an ion-permeable isoelectric barriers having pI values different from the pI values of the ampholytic sample component and the isoelectric buffer.

Another aspect provides for selecting a second isoelectric buffer having a pI value different from the pI values of the ampholytic sample component, the first isoelectric buffer, and the isoelectric barrier. Another aspect provides for trapping a first ampholytic sample component and a second ampholytic sample component in their non-isoelectric states on either side of an isoelectric barrier by executing an electrophoretic separation.

In a first aspect, the present invention provides a method for separating an ampholytic component by electrophoresis, the method comprising:

placing a sample containing an ampholytic component having a pI value in an electrophoresis separation system comprising an anolyte having a pH and a catholyte having a pH, the catholyte pH being higher than the anolyte pH, one or more ion-permeable barriers disposed between the anolyte and catholyte wherein at least one of the barriers is anisoelectric barrier having a pI value which is higher than the anolyte pH and lower than the catholyte pH;

providing an isoelectric buffer having a pI value higher than the pH of the anolyte and lower than the pH of the catholyte and different from the pI value of the ampholytic sample component and different from the pI value of an ion-permeable isoelectric barrier; and

exposing the sample to an electric potential so as to trap the ampholytic sample component in a non-isoelectric state in the presence of the isoelectric buffer in the electrophoresis system.

Preferably, the electrophoretic separation system comprises an anode compartment containing an anode and adapted to receive the anolyte, a cathode compartment containing a cathode and adapted to receive the catholyte, and one ion-permeable barrier in the form of an ion-permeable isoelectric barrier having a pI value disposed between the anode compartment and the cathode compartment.

Preferably, the electrophoretic separation system further comprises first and second ion-permeable barriers disposed between the anode compartment and the cathode compartment forming a first separation compartment between the anode compartment and cathode compartment.

In a preferred form, the first and second ion-permeable barriers are isoelectric barriers each having a defined but different pI.

The sample can be provided to at least one of the sample compartment, anode compartment or the cathode compartment. In one preferred form, the sample is provided to a sample compartment and the ampholytic component is separated in a non-isoelectric state in the sample compartment. Alternatively, the ampholytic component can be obtained in a non-isoelectric state in the sample compartment or the cathode compartment or the anode compartment.

It will be appreciated that the sample can contain two or more ampholytic components which are obtained in non-isoelectric states in the sample compartment or the cathode compartment or the anode compartment or two or more of the sample compartment, the anode compartment or the cathode compartment.

The electrophoretic separation system may further comprise a third ion-permeable barrier disposed between the anode compartment and the cathode compartment forming a second separation compartment adjacent the first separation compartment, the system having first ion-permeable isoelectric barrier having a first pI value and a second ion-permeable isoelectric barrier having a second pI value different from the first pI value of the first ion-permeable isoelectric barrier.

Preferably, all the ion-permeable barriers are isoelectric barriers each having a defined but different pI. It will be appreciated that any one or more of the barriers can be isoelectric barriers.

In this form, the sample can be provided to the first sample compartment, the second sample compartment, both the first and second sample compartments, or the anode compartment or the cathode compartment. The ampholytic component can be obtained in a non-isoelectric state in the first sample compartment or in the second sample compartment, or in the anode compartment, or in the cathode compartment.

The sample can contain two or more ampholytic components which are obtained in a non-isoelectric state in the first sample compartment or in the second sample compartment or the anode compartment or the cathode compartment, or in each of the first sample compartment and second sample compartment.

In another preferred form, the electrophoretic separation system comprises a plurality of ion-permeable barriers disposed between the anode compartment and the cathode compartment forming a plurality of separation compartments disposed between the anode and cathode compartments, wherein two or more ion-permeable barriers are ion-permeable isoelectric barriers each having a different pI value. Three or more of the ion-permeable barriers can be ion-permeable isoelectric barriers each having a different pI value. It will be appreciated that all the ion-permeable barriers can be ion-permeable isoelectric barriers each having a different pI value.

The method can include providing a first isoelectric buffer and a second isoelectric buffer each having a different pI value to at least two of the sample compartments or the anode compartment or the cathode compartment.

The ion-permeable barriers can be selected from any suitable material or substance such as immiscible liquids, porous solids, non-ionic membranes, membranes, isoelectric membranes, hydrogel membranes, gels, and hydrogels.

The present inventor has found that hydrogel membranes are preferably polyether sulfone-based, poly(vinyl) alcohol-based or polyacrylamide-based. It will be appreciated that any other type of hydrogel membrane can be used for the present invention.

Preferably, the non-ionic membranes are supported membranes such as cross-linked polyacrylamide and poly(vinyl) alcohol supported on glass fiber, filter paper, polymeric mesh, and paper. The ion-permeable barriers can be porous frits such as glass frits, ceramic frits, and polymeric frits.

In one preferred form, the ion-permeable barriers can substantially restrict the passage of molecules having a size or hydrated ion-radius greater than a predetermined size.

The ion-permeable isoelectric barriers are preferably isoelectric porous solids, isoelectric membranes, and isoelectric gels. More preferably, the ion-permeable isoelectric barriers are isoelectric membranes.

The ion-permeable barriers preferably prevent substantial convective mixing of solutes between the barriers. In use, preferably there is substantially no convective mixing between the compartments.

The isoelectric buffer can be provided to at least one of a sample compartment, the anode compartment and the cathode compartment. Preferably, the pI value of the isoelectric buffer differs by at least about 0.001 pH units from the pI value of the ampholytic sample component. The absolute value of the difference between the pI value and the pKa value closest to the pI value of the isoelectric buffer preferably is less than about 1.5.

The isoelectric buffer can be any suitable buffer which will allow an ampholytic component to be obtained in a substantially non-isoelectric state. Suitable buffers include, but not limited to, iminodiacetic acid, N-methylimino diacetic acid, aspartic acid, glutamic acid, glycyl-aspartic acid, m-aminobenzoic acid, histidyl-glycine, histidyl-histidine, histidine, 1,2-diaminopropionic acid, ornithine, lysine, lysil-lysine, and arginine. Preferably, the isoelectric buffer is glutamic acid or lysine. When two isoelectric buffers are used, the present inventor has found that glutamic acid and lysine are particularly suitable.

The method can further include providing a non-ionic solubilizing agent. Preferably, the solubilizing agent is a non-ionic detergent. Examples include, but not limited to, TWEEN™ 20, Brij™30, TRITON™ X-100, or NDSB-195.

The method is particularly suitable for the separation of ampholytic sample components such as proteins, polypeptides, oligopeptides, amino phenols, amino phosphonic acids, and amino acids. Preferably, the ampholytic sample component is a protein.

The present invention overcomes problems with prior art isoelectric separations that require the component to be separated in its isoelectric state in that the electrophoretic separation is much faster and there are fewer problems with the solubility of the component.

A further advantage of the present invention is that it can be carried out in any electrophoresis separation system that can be adapted for isoelectric separations. The method does not require a special apparatus and results in the more efficient use of known electrophoresis separation systems.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In order that the present invention may be more clearly understood, preferred forms will be described with reference to the following drawings and examples.

### BRIEF DESCRIPTION OF THE DRAWINGS

FIG. 1 is a schematic diagram of an isoelectric trapping (IET) unit (Prior Art) (Gradipore, Australia) that may be used to practice embodiments of the present application;

FIG. 2 is a schematic diagram indicating the characteristics of representative solutions in the compartments of the IET unit shown in FIG. 1 at the beginning of the separation process according to the present application;

FIG. 3 is a full-column imaging UV absorbance detector trace of a chicken egg white sample analyzed by capillary isoelectric focusing as described in Example 1;

FIG. 4 is a full-column imaging UV absorbance detector trace of aliquots removed at the end of an IET separation from the cathodic and anodic separation compartments of the IET unit that is shown in FIG. 1 and was operated as described in Example 2, analyzed by capillary isoelectric focusing as described in Example 1;

FIG. 5 is a full-column imaging UV absorbance detector trace of aliquots removed at the end of the IET separation from the cathodic and anodic separation compartments of the IET unit that is shown in FIG. 1 and was operated as described in Example 3, analyzed by capillary isoelectric focusing as described in Example 1;

FIG. 6 is a full-column imaging UV absorbance detector trace of the aliquot removed from the cathodic and anodic separation compartments of the IET unit that is shown in FIG. 1 and was operated as described in Example 4, at the end of the IET separation of the sample obtained from the anodic separation compartment in Example 3, analyzed by capillary isoelectric focusing as described in Example 1;

FIG. 7 is a full-column imaging UV absorbance detector trace of aliquots removed at the end of the IET separation from the cathodic and anodic separation compartments of the IET unit that is shown in FIG. 1 and was operated as described in Example 5, analyzed by capillary isoelectric focusing as described in Example 1.

FIG. 8 is a UV absorbance detector trace recorded during a pressure mediated capillary electrophoretic separation of aliquots removed at the beginning and end of the IET separation from the separation compartment of the IET unit that was operated as described in Example 6.

FIG. 9 is a UV absorbance detector trace recorded during a pressure mediated capillary electrophoretic separation of aliquots removed at the beginning and end of the IET separation from the separation compartment of the IET unit that was operated as described in Example 7.

FIG. 10 is a full-column imaging UV absorbance detector trace of aliquots removed at the end of the IET separation after eight passes from the cathodic and anodic separation compartments of the IET unit that is shown in FIG. 1 and was operated as described in Example 8, analyzed by capillary isoelectric focusing as described in Example 1;

### MODE(S) FOR CARRYING OUT THE INVENTION

Embodiments for altering the initial composition of a sample mixture having an ampholytic sample component according to the present application are described in non-limiting detail below.

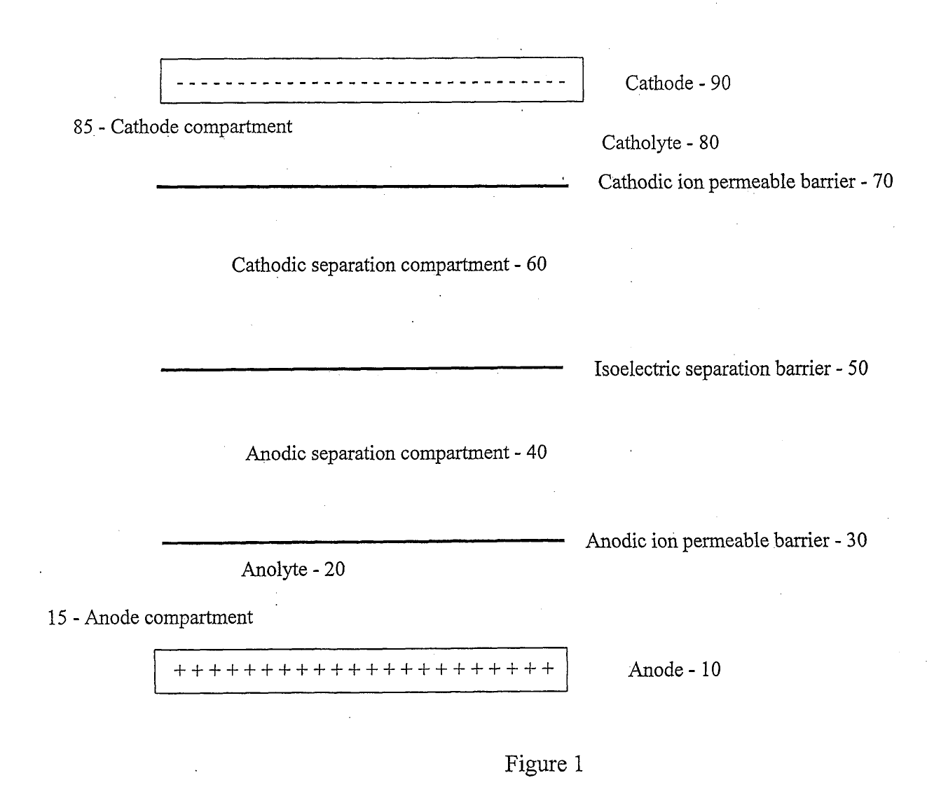

FIG. 1 refers to an isoelectric trapping (IET) unit known in the art that may be used to practice embodiments described in the application. In this IET separation unit (Gradipore, Australia), anode 10 is located in anode compartment 15. Anolyte 20 occupies anode compartment 15. Anolyte 20 is an acid solution, for example, it is an aqueous solution of phosphoric acid (H₃PO₄). Of course, other suitable acid solutions known in the art may also be used. Anodic ion-permeable barrier 30 separates anode compartment 15 from adjacent anodic separation compartment 40. Ion-permeable barrier 30 allows passage of ions but substantially prevents convective mixing of the contents of anode compartment 15 and anodic separation compartment 40. Ion-permeable barrier 30 is also commonly referred to as an ion-permeable, anti-convective barrier or restriction membrane. While in this example anodic ion-permeable barrier 30 is an ion-permeable, anti-convective, non-isoelectric barrier, it may also be an ion-permeable, anti-convective, isoelectric barrier, or any other barrier that allows for passage of ions and prevents convective mixing of the contents of anodic separation compartment 40 and anode compartment 15. Ion-permeable isoelectric separation barrier 50, having adequate buffering and titrating capacity at its pI value, separates anodic separation compartment 40 and cathodic separation compartment 60 and substantially prevents convective mixing of the contents of compartments 40 and 60. Isoelectric separation barrier 50 may be an isoelectric membrane, immobilized isoelectric gel, isoelectric gateway, or any other isoelectric barrier known in the art that has adequate buffering and titrating capacity at its pI value.

Cathodic ion-permeable barrier 70 separates cathodic separation compartment 60 and cathode compartment 85 that contains catholyte 80. Cathodic ion-permeable barrier 70 allows passage of ions, but substantially prevents convective mixing of the contents of cathodic separation compartment 60 and cathode compartment 85. Cathodic ion-permeable barrier 70 is also commonly referred to as an ion-permeable, anti-convective barrier or a restriction membrane. While in this example cathodic ion-permeable barrier 70 is an ion-permeable, anti-convective, non-isoelectric barrier, it may also be an ion-permeable, anti-convective, isoelectric barrier or any other barrier that allows for passage of ions and prevents convective mixing of the contents of cathodic separation compartment 60 and cathode compartment 85. Catholyte 80 is a base solution, for example, it is an aqueous solution of sodium hydroxide (NaOH). Other suitable base solutions known in the art may also be used. Cathode compartment 85 contains cathode 90.

While FIG. 1 is merely one example of a suitable IET separation unit, other IET separation unit configurations may also be used. The criteria for selecting the elements of a suitable IET unit according to fundamental IET principles are well known in the art (e.g., Faupel et al., U.S. Pat. No. 5,082,548 which are herein incorporated by reference). For example, only one separation compartment between anodic ion-permeable barrier 30 and cathodic ion-permeable barrier 70 may be used or more than two separation compartments between anodic ion-permeable barrier 30 and cathodic ion-permeable barrier 70 may be used. Furthermore, anodic ion-permeable barrier 30 and cathodic ion-permeable barrier 70 may be of similar construction or different. For example, they may be ion-permeable, anti-convective, non-isoelectric barriers or ion-permeable, anti-convective, isoelectric barriers, or any other barriers known in the art such that barriers 30 and 70 are ion-permeable and substantially prevent convective mixing of the contents of the adjacent compartments they separate.

While FIG. 1 shows an electrophoresis unit or system having three ion-permeable barriers, the present invention may be performed in a system having only one isoelectric barrier separating the anode compartment for the cathode compartment. In this form, the sample and isoelectric buffer can be provided to one or more of the anode and cathode compartments and the component separated in a non-isoelectric state in either of the anode and cathode compartments.

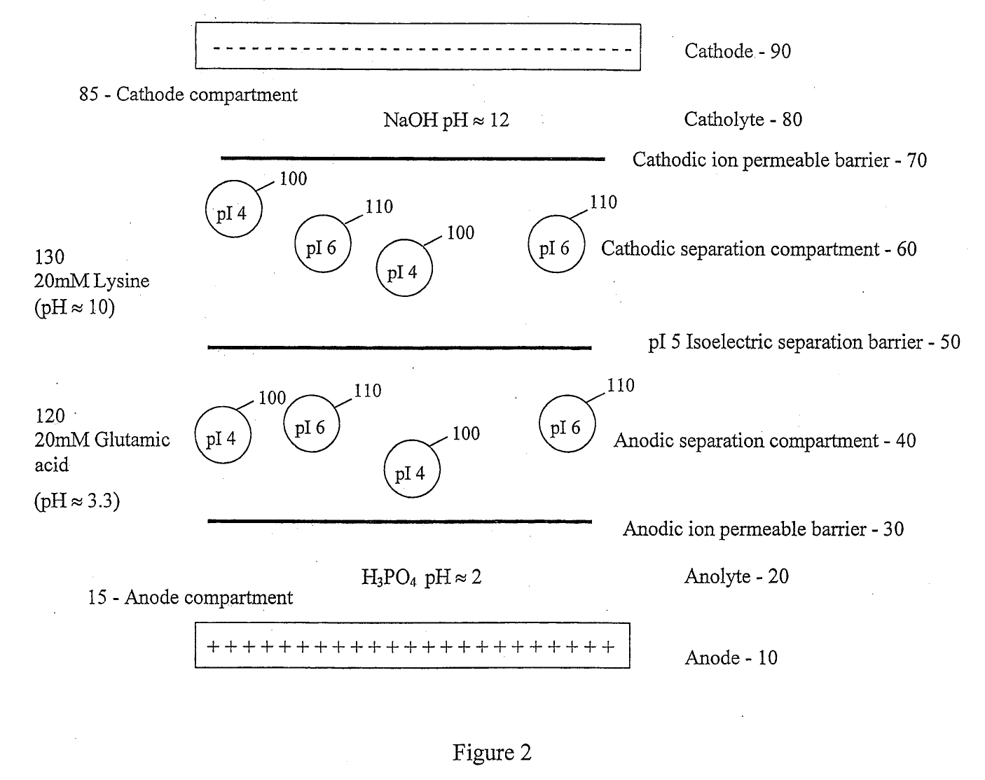

FIG. 2 is a schematic diagram indicating the characteristics of representative solutions in the compartments of the IET unit shown in FIG. 1 at the beginning of an IET process according to the present application. One feature is the use of an isoelectric, buffer in a separation compartment of an IET unit wherein the isoelectric buffer has adequate buffering and titrating capacity and conductivity at its pI value. For example, anodic separation compartment 40 contains a mixture of ampholytic sample components 100 and 110 and first isoelectric buffer 120 wherein first isoelectric buffer has adequate buffering and titrating capacity and conductivity at its pI value, and cathodic separation compartment 60 contains a mixture of ampholytic sample components 100 and 110 and second isoelectric buffer 130 wherein second isoelectric buffer 130 also has adequate buffering capacity, titrating capacity and conductivity at its pI value. The pI value of isoelectric separation barrier 50 is selected to be different from the pIvalues of first ampholytic sample component 100 and second ampholytic sample component 110. For example, in FIG. 2, isoelectric separation barrier 50 is selected to have a pI value between the pIvalues of ampholytic sample components 100 and 110.

The pI values of first and second isoelectric buffers 120 and 130 are selected to be different both from the pIvalues of first and second ampholytic sample components 100 and 110, the pI value of isoelectric separation barrier 50, and the pH value of anolyte 20 and catholyte 80 such that at the end of the IET separation process, first and second ampholytic sample components will be present in their non-isoelectric state. In FIG. 2, ampholytic sample component 100 is, e.g., a protein with pI=4, while ampholytic sample component 110 is, e.g., a protein with pI=6. Isoelectric separation barrier 50 has a pI value of 5, first isoelectric buffer 120 has a pI value of about 3.3, second isoelectric buffer 130 has a pI value of about 10, and anolyte 20 has a pH value of about 2 and catholyte 80 has a pI value of about 12. In this example, isoelectric buffer 120 is a 20 mM glutamic acid solution, isoelectric buffer 130 is a 20 mM lysine solution, anolyte 20 is an aqueous solution of phosphoric acid, and catholyte 80 is an aqueous solution of sodium hydroxide.

According to well known IET precepts, application of the electric potential between anode 10 and cathode 90 forces all ampholytic components to migrate into the respective compartments of the IET unit. At the end of the IET separation process, all ampholytic components with pI values higher than the pH value of anolyte 20 and lower than the pI value of isoelectric separation barrier 50 are trapped in anodic separation compartment 40 (i.e., first ampholytic sample component 100 and isoelectric buffer 120 are trapped in anodic separation compartment 40), and all ampholytic components with pI values lower than the pH value of catholyte 80 and higher than the pI value of isoelectric separation barrier 50 are trapped in cathodic separation compartment 60 (i.e., second ampholytic sample component 110 and isoelectric buffer 130 are trapped in cathodic separation compartment 60). However, due to the presence of isoelectric buffer 120 in anodic separation compartment 40, first ampholytic sample component 100 is obtained at the end of the IET process in its non-isoelectric state. Also, due to the presence of isoelectric buffer 130 in cathodic separation compartment 60, second ampholytic sample component 110 is obtained at the end of the IET process in its non-isoelectric state.

Practitioners in the art will appreciate that more than two ampholytic sample components may be present in the sample at any one time. Practitioners in the art will also appreciate that first isoelectric buffer 120 and second isoelectric buffer 130 may be introduced into compartments 40 and/or 60 separate from each other (e.g., isoelectric buffer 120 may be introduced into anodic separation compartment 40 and isoelectric buffer 130 may be introduced into cathodic separation compartment 60), individually mixed with the sample (e.g., isoelectric buffer 120 and a mixture of ampholytic sample components 100 and 110 may be introduced into anodic separation compartment 40, and isoelectric buffer 130 and a mixture of ampholytic sample components 100 and 110 may be introduced into cathodic separation compartment 60), mixed with each other (e.g., isoelectric buffers 120 and 130 may both be introduced into compartments 40 and/or 60), or mixed with each other and the sample (e.g., isoelectric buffer 120, isoelectric buffer 130, ampholytic sample components 100 and 110 may be introduced into compartments 40 and/or 60). Practitioners in the art will further appreciate that there may be more than two ampholytic sample components, more than two separation compartments, more than two isoelectric buffers and more than one isoelectric separation barrier at any one time.

Further, practitioners in the art will appreciate that the sequence or order of introduction of the sample and isoelectric buffer(s) to the separation unit may vary, that is, the sample or buffers may be introduced to the separation unit together or in any sequence.

As a non-limiting example, a mixture of several ampholytic sample components having different pIvalues may be separated into two parts. In this example, at the end of an IET separation process according to the present application, first ampholytic sample component 100 may comprise a mixture of ampholytic sample components having pI values lower than the pI value of isoelectric separation barrier 50 while second ampholytic sample component 110 may comprise a mixture of ampholytic sample components having pIvalues higher than the pI value of isoelectric separation barrier 50. Ampholytic sample components 100 and 110 may be any ampholytic sample components that can be separated according to isoelectric focusing or isoelectric trapping principles.

In FIG. 2, isoelectric separation barrier 50 is selected to have its pI value higher than the pI value of first ampholytic sample component 100. First isoelectric buffer 120 is selected to have its pI value lower than the pI value of isoelectric separation barrier 50. Although first isoelectric buffer 120 has a pI value lower than isoelectric separation barrier 50, first isoelectric buffer 120 may have a pI value higher or lower than the pI value of first ampholytic sample component 100 because at the end of the IET separation both will force first ampholytic sample component 100 into a non-isoelectric state. For example, in FIG. 2, isoelectric buffer 120 has a pI value lower than the pI value of first ampholytic sample 100. However, isoelectric buffer 120 may also have a pI value higher than the pI value of first ampholytic sample component 100.

Similarly, in FIG. 2, isoelectric separation barrier 50 is selected to have its pi value lower than the pI value of second ampholytic sample component 110. Second isoelectric buffer 130 is selected to have its pI value higher than the pI value of isoelectric separation barrier 50. Although second isoelectric buffer 130 has a pI value higher than the pI value of isoelectric separation barrier 50, second isoelectric buffer 130 may have a pI value higher or lower than the pI value of second ampholytic sample component 110, because at the end of the IET separation both will force second ampholytic sample component 110 into a non-isoelectric state. For example, in FIG. 2, second isoelectric buffer 130 has a pI value higher than the pI value of second ampholytic sample component 110. However, isoelectric buffer 130 may also have a pI value lower than the pI value of second ampholytic sample component 110.

In another embodiment, the pI value of isoelectric buffer 120 is higher than the pI value of isoelectric barrier 50 and the pI value of isoelectric buffer 130 is lower than the pI value of isoelectric barrier 50. Although the pI value of isoelectric buffer 120 is higher than the pI value of isoelectric barrier 50, the pI value of isoelectric buffer 120 is lower than the pI value of second ampholytic sample component 110. Similarly, although the pI value of isoelectric buffer 130 is lower than the pI value of isoelectric separation barrier 50, the pI value of isoelectric buffer 130 is higher than the pI value of first ampholytic sample component 100. In another embodiment, the pI value of isoelectric buffer 120 is higher than the pI value of isoelectric barrier 50 and higher than the pI value of second ampholytic sample component 110, and the pI value of isoelectric buffer 130 is lower than the pI value of isoelectric separation barrier 50 and lower than the pI value of a first ampholytic sample component 100.

While FIG. 2 uses two isoelectric buffers, the use of only one isoelectric buffer also practices embodiments of the application. For example, isoelectric buffer 120 may be used without using isoelectric buffer 130. Similarly, isoelectric buffer 130 may be used without using isoelectric buffer 120. For example, the use of a single isoelectric buffer may be advantageous when the sample contains several ampholytic components of dissimilar pI values.

As a non-limiting example of this principle of using only one isoelectric buffer, isoelectric buffer 120 is selected to have a pI value that insures that at the end of the IET separation, isoelectric buffer 120 is present in the same separation compartment as the selected ampholytic sample component (e.g., first ampholytic sample component 100) and renders that sample component non-isoelectric. For example, when first ampholytic sample component 100 has a pI value higher than the pI value of isoelectric separation barrier 50, isoelectric buffer 120 is also selected to have a pI value higher than isoelectric separation barrier 50. Similarly, when first ampholytic sample component 100 has a pI value lower than isoelectric separation barrier 50, isoelectric buffer 120 is selected to have a pI value lower than the pI value of isoelectric separation barrier 50. While isoelectric buffer 120 has a pI value different from the pI value of first ampholytic sample component 100, the pI value of isoelectric buffer 120 may be higher or lower than the pI value of first ampholytic sample component 100. In one embodiment, isoelectric buffer 120 is selected to have a pI value higher than the pI value of first ampholytic sample component 100. In another embodiment, isoelectric buffer 120 is selected to have a pI value lower than the pI value of first ampholytic sample component 100.

In another non-limiting embodiment, isoelectric buffer 120 may be selected to have a pI value that insures that at the end of the IET separation, isoelectric buffer 120 is present in a different separation compartment than first and second ampholytic sample components 100 and 110. In this manner, ampholytic sample components may be obtained on one side of isoelectric separation barrier 50 while non-electrolyte sample components remain on the other side of isoelectric separation barrier 50. For example, in one embodiment, a sample solution contains ampholytic sample components 100 and 110 that have different pI values, along with non-electrolyte sample component 115. The pI value of isoelectric separation barrier 50 is selected such that ampholytic sample components 100 and 110 are located on the same side of isoelectric separation barrier 50 at the end of the IET separation process. For example, in this embodiment, the pI value of isoelectric barrier 50 is selected to be lower than the pI value of both ampholytic sample components 100 and 110. Isoelectric buffer 120 is selected to have a pI value such that at the end of the IET separation process isoelectric buffer 120 is located on the other side of isoelectric separation barrier 50 than ampholytic sample components 100 and 110. For example, in this embodiment, isoelectric buffer 120 is selected to have a pI value lower than both the pI values of ampholytic sample components 100 and 110 and isoelectric separation barrier 50. Cathodic separation compartment 60 is filled with isoelectric buffer 120, while anodic separation compartment 40 may be filled with the sample solution alone, or with a mixture of the sample solution and isoelectric buffer 120. At the end of the IET separation process, anodic separation compartment contains non-electrolyte sample component 115 and isoelectric buffer 120, the latter providing adequate conductivity in anodic separation compartment 40. At the end of the IET separation process, cathodic separation compartment 60 contains a mixture of ampholytic sample components 100 and 110, which provides adequate conductivity in cathodic separation compartment 60.

When the sample is placed only into one of the separation compartments of an IET unit, it may be advantageous, though not necessary, to fill the other separation compartment with isoelectric buffer 120 rather than water, because isoelectric buffer 120 improves the potential distribution across the IET unit and leads to a faster IET separation.

Suitable isoelectric buffers 120 and 130 have adequate buffering and titrating capacity and conductivity in the vicinity of their pI values. In order for an isoelectric buffer to have adequate buffering capacity and adequate conductivity at its pI value, it is preferable that the difference between its pI value and its closest pKa value is less than 1, preferably less than 0.75 and more preferably less than 0.5. For example, suitable isoelectric buffers include N-methylimino diacetic acid, iminodiacetic acid, aspartic acid, glutamic acid, glycyl-aspartic acid, m-aminobenzoic acid, histidyl-glycine, histidyl-histidine, histidine, 1,2-diaminopropionic acid, ornithine, lysine, lysil-lysine, and arginine. Practitioners in the art will appreciate that other suitable isoelectric buffers having adequate buffering and titrating capacities and conductivities may also be used.

While FIG. 2 illustrates a single binary separation, other embodiments practice sequential binary separations. The isoelectric buffers selected for each sequential IET separation may be the same or different. For example, at the end of a first sequential IET separation, isoelectric buffer 120 may be used to contain both first ampholytic sample components 100 and 110. If the desired target of a second sequential IET separation is first isoelectric sample component 100, and it is desired to be obtained in a solution of isoelectric buffer 135, isoelectric separation barrier 50 for the second sequential separation may be selected to have its pI value between the pI values of first and second isoelectric sample components 100 and 110, isoelectric buffer 120 is selected to have its pI value greater than the pI values of both isoelectric sample components 100 and 110 and isoelectric separation barrier 50. In one embodiment, anodic and cathodic separation compartments 40 and 60 may be filled with a mixture of isoelectric sample components 100 and 110 and isoelectric buffers 120 and 135. In another embodiment, anodic separation compartment 40 may be filled with isoelectric buffer 135 and cathodic separation compartment 60 may be filled with a mixture of isoelectric sample components 100 and 110 and isoelectric buffers 120 and 135. In another embodiment, anodic separation compartment 40 may be filled with isoelectric buffer 135 and cathodic separation compartment 60 may be filled with a mixture of isoelectric sample components 100 and 110 and isoelectric buffer 120. In another embodiment, anodic separation compartment 40 may be filled with isoelectric buffer 135 and cathodic separation compartment 60 may be filled with a mixture of isoelectric sample components 100 and 110 and isoelectric buffers 120 and 135.

In other embodiments, fresh isoelectric buffer may be added in any sequential separation step or isoelectric buffer may be carried over from a previous separation step. Other embodiments practice simultaneous multiple-cut separations (e.g., conducted on an IsoPrime™ unit (Amersham-Pharmacia)) with multiple isoelectric buffers.

While the presence of an isoelectric buffer typically enhances the solubility of an ampholytic sample component by rendering it non-isoelectric, suitable solubilizing agents used in IEF systems may also be used in the present application. For example, non-ionic and zwiterrionic solubilizing agents known in the art may also be used to increase solubility of the sample components. For example, TWEEN™ 20, Brij™ 30, TRITON™ X-100 (Aldrich) or any other suitable non-ionic solubilizing agents may also be used. Similarly, NDSB-195™ (Toronto Research Chemicals, North York, ON, Canada) or any other suitable zwiterrionic solubilizing agents known in the art may be used.

To assist in understanding the present application, the following examples are included and describe the results of a series of experiments. The following examples relating to this invention should not be construed to specifically limit the invention or such variations of the invention, now known or later developed, which fall within the scope of the invention as described and claimed herein.

### EXAMPLES

### Example 1

Full-Column UV Absorbance Imaging Capillary Isoelectric Focusing Separation of Chicken Egg White Sample.

Chicken egg white, diluted at a rate of 1 to 25 in deionized water was used as a sample that contains ampholytic components. Full-column UV absorbance imaging capillary isoelectric focusing on an iCE280 instrument (Convergent Bioscience, Toronto, Canada) was used to analyze the chicken egg white sample. The fused silica separation capillary was 5 cm long, its internal diameter was 75 micrometer.

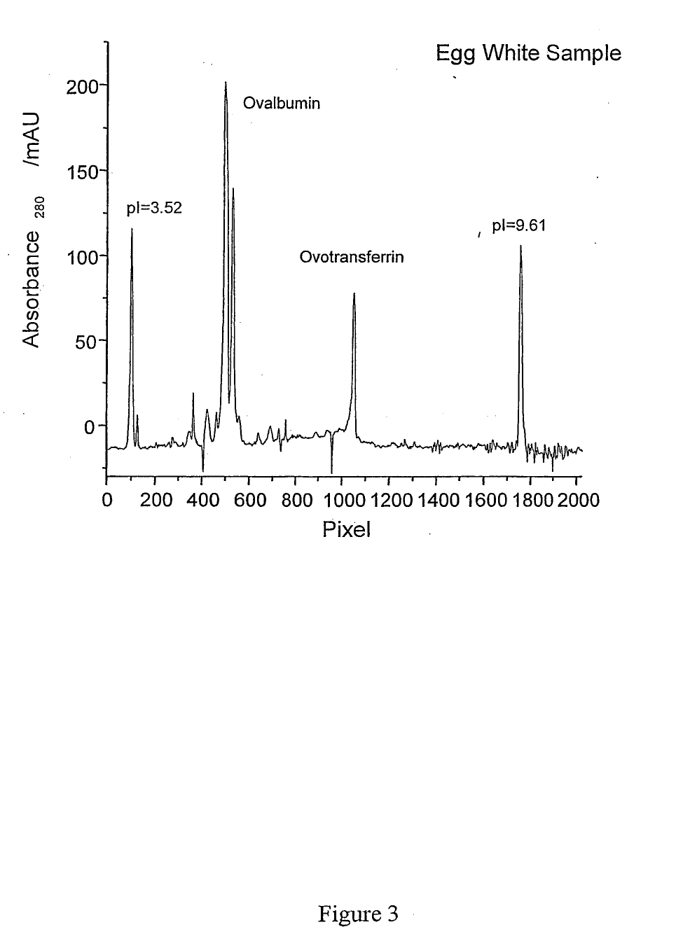

The focusing medium contained 8% carrier ampholytes (Pharmalyte 3-10, Amersham-Pharmacia) covering a pH 3-10 range, in an aqueous, 0.28% methylcellulose solution (avg. m.w.=80,000, Aldrich). 75 microliters of the sample to be analyzed was mixed with 150 microliters of the focusing medium. An aliquot of this solution was filled into the separation capillary and focused for 5 minutes at 3,000 V. Dansyl phenylalanine (approximate pI=3.52) and terbutaline (approximate pI=9.61) were used as pI markers. FIG. 3 shows the full-column UV absorbance imaging capillary IEF electropherogram of the chicken egg white sample. Ovalbumin focused in a region of the capillary that corresponds to pixels 450 to 650. Ovotransferrin focused in a region of the capillary that corresponds to pixels 1050 to 1150.

### Example 2

Preparative-Scale Separation of a Chicken Egg White Sample by a Known IET Method (Prior Art).

In order to compare the present application with a known IET method, the preparative-scale separation of a sample that contains ampholytic components was carried out using a known IET method under the conditions described below.

This separation representing the prior art was obtained on an IET unit produced by Gradipore, Australia. The IET cartridge (U.S. Pat. No. 6,328,869) contained anodic ion-permeable barrier 30 made of a 5,000 D nominal cut-off polyether sulfone membrane (Gelman), anodic separation compartment 40, pI=4.7 isoelectric separation barrier 50 (Gradipore) prepared from Immobiline™ chemicals (Amersham-Pharmacia), acrylamide and N-N′-methylene bis-acrylamide (Amersham-Pharmacia), cathodic separation compartment 60, and cathodic ion-permeable barrier 70 made of a 5,000 D nominal cut-off polyether sulfone membrane (Gelman). 80 mL of a 50 mM phosphoric acid solution (approximate pH=1.8) was circulated through anode compartment 15 at a flow-rate of 2 L/min. 80 mL of a 50 mM sodium hydroxide solution (approximate pH=12.4) was circulated through cathode compartment 85 at a flow-rate of 2 L/min. 40 mL each of a filtered chicken egg-white solution (containing egg white diluted with deionized water at a rate of 1 to 25) was circulated through anodic and cathodic separation compartments 40 and 60, respectively, at a flow rate of 20 mL/min. Anode 10 was placed into anode compartment 15, and cathode 90 was placed into cathode compartment 85. The applied potential was initially 250 V, producing a current of 400 mA. After 10 passes of the chicken egg white solution through the separation compartments, the current dropped to 37 mA. The potential was then increased to 500 V, which produced a current of 74 mA. The separation was practically complete after 7 more passes, as indicated by the finishing current leveling off at 25 mA, resulting in a total separation time of 40 minutes. Aliquots were taken from anodic and cathodic separation compartments 40 and 60 after each pass of the chicken egg white solution and analyzed by the full-column UV absorbance imaging capillary IEF instrument as described in Example 1.

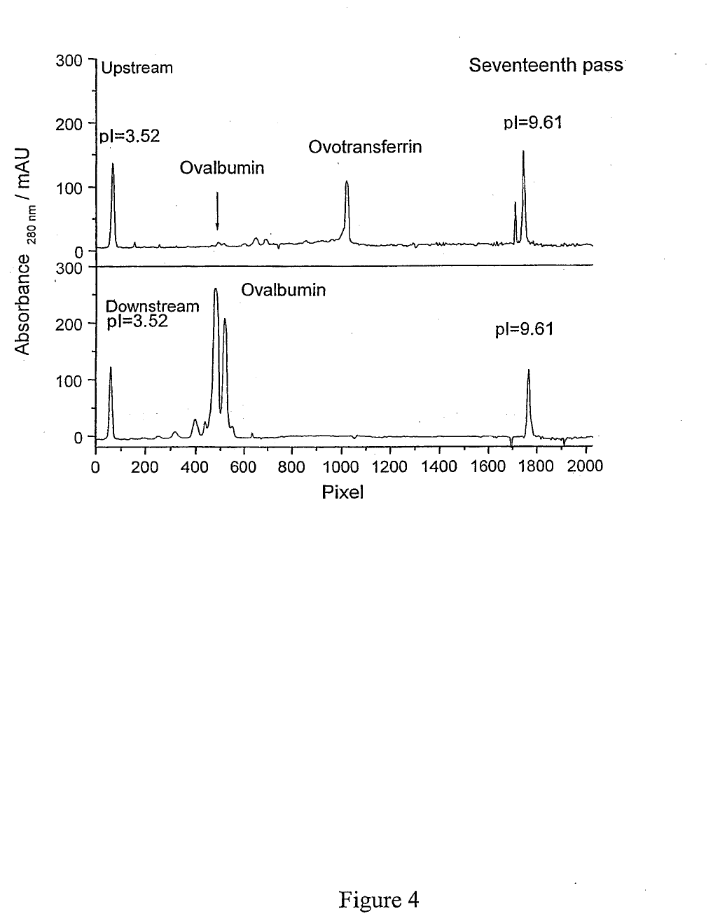

As a result of this IET separation, proteins with pI values lower than 4.7, such as ovalbumin (approximate pI=4.6), accumulated in anodic separation compartment 40 on the anodic side of pI=4.7 isoelectric separation barrier 50 (bottom panel in FIG. 4, labeled “Downstream”). Proteins with pI values greater than 4.7, such as ovotransferrin (approximate pI=6.1) accumulated in cathodic separation compartment 60 on the cathodic side of isoelectric separation barrier 50 (top panel in FIG. 4, labeled “Upstream”). Practically all of ovalbumin and the lower pIproteins transferred into anodic separation compartment 40. Visual inspection of the interior of the separation cartridge at the end of this IET separation revealed the presence of precipitated protein in anodic separation compartment 40.

### Example 3

Preparative-Scale Separation of a Chicken Egg White Sample by an IET Separation

The preparative-scale separation of a sample that contains ampholytic components was carried out in the presence of two isoelectric buffers according to the present application under the conditions described below.

The IET separation according to the present application was obtained on the IET unit used in Example 2. The IET cartridge (U.S. Pat. No. 6,328,869) contained anodic ion-permeable barrier 30 made of a 5,000 D nominal cut-off polyether sulfone membrane (Gelman), anodic separation compartment 40, pI=4.8 isoelectric separation barrier 50 (Gradipore) prepared from Immobiline™ chemicals (Amersham-Pharmacia), acrylamide and N-N′-methylene bis-acrylamide (Amersham-Pharmacia), cathodic separation compartment 60, and cathodic ion-permeable barrier 70 made of a 5,000 D nominal cut-off polyether sulfone membrane (Gelman). 80 mL of a 50 mM phosphoric acid solution (approximate pH=1.8) was circulated through anode compartment 15 at a flow rate of 2 L/min. 80 mL of a 50 mM sodium hydroxide solution (approximate pH=12.4) was circulated through cathode compartment 85 at a flow rate of 2 L/min. 40 mL each of a filtered chicken egg white solution (containing chicken egg white dissolved in 20 mM glutamic acid and 20 mM lysine, respectively, at a dilution rate of 1 to 25) was circulated through anodic and cathodic separation compartments 40 and 60, respectively, at a flow rate of 20 mL/min. Anode 10 was placed into anode compartment 15, and cathode 90 was placed into cathode compartment 85. The applied potential was 250 V that initially produced a current of 480 mA. The separation was practically complete after 10 passes of the chicken egg white solution through the separation compartments, indicated by the finishing current leveling off at 53 mA, resulting in a total separation time of 25 minutes. Aliquots were taken from anodic and cathodic separation compartments 40 and 60 after each pass and analyzed by the full-column UV absorbance imaging capillary IEF instrument as described in Example 1.

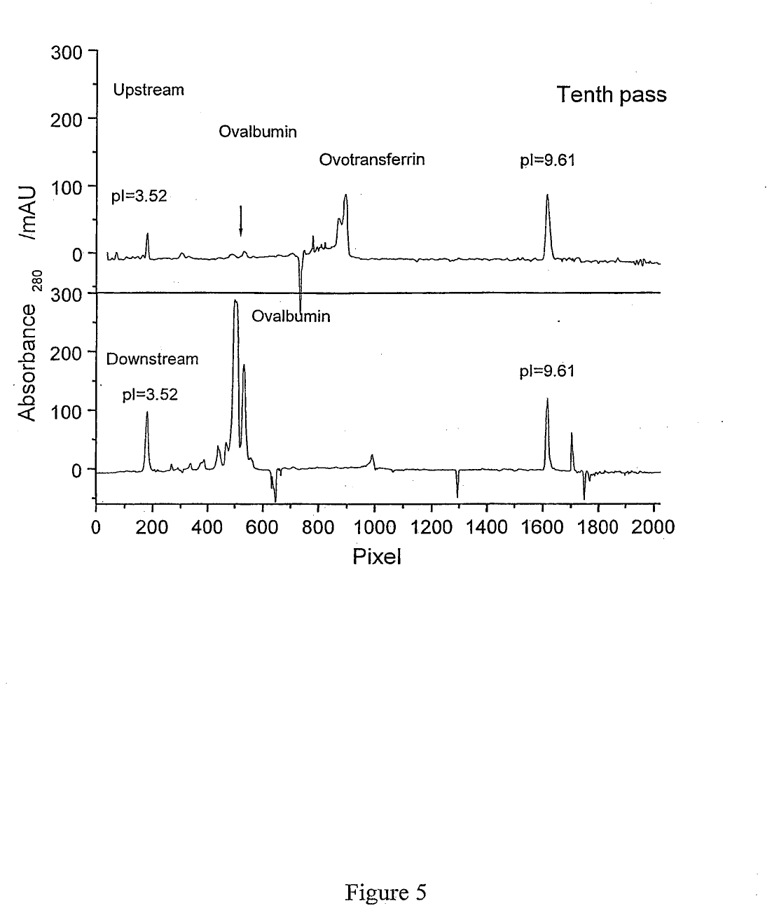

As a result of the IET separation according to the present application, proteins with pI values lower than 4.8, such as ovalbumin (approximate pI=4.6), accumulated in anodic separation compartment 40 on the anodic side of pI=4.8 isoelectric separation barrier 50 (bottom panel in FIG. 5 labeled “Downstream”). Proteins with pI values greater than 4.8, such as ovotransferrin (approximate pI=6.1) accumulated in cathodic separation compartment 60 on the cathodic side of isoelectric separation barrier 50 (top panel in FIG. 5 labeled “Upstream”). Practically all of ovalbumin and the lower pI proteins transferred into anodic separation compartment 40. Visual inspection of the interior of the separation cartridge at the end of this IET separation indicated that protein precipitation did not occur either in anodic separation compartment 40 or cathodic separation compartment 60.

Thus, effectively the same separation was achieved in Example 3 as in Example 2, but the separation required a shorter period of time and a smaller number of passes of these solutions through the IET cartridge despite the fact that the potential applied over the course of the separation was lower. Additionally, one of the main limitations of known IET separations, i.e., that the low solubility of the ampholytic sample components recovered in their isoelectric state causes their precipitation, has been mitigated.

### Example 4

Preparative-Scale Sequential Binary Separation of a Chicken Egg White Sample by an IET Separation

The preparative-scale, sequential binary separation of a sample that contains ampholytic components was carried out in the presence of two isoelectric buffers according to the present application under the conditions described below to obtain a fraction from ampholytic components with closely spaced pI values.

In this example, the sample recovered from anodic separation compartment 40 in Example 3 was again subjected to IET separation using an IET cartridge (U.S. Pat. No. 6,328,869) that contained anodic ion-permeable barrier 30 made of a 5,000 D nominal cut-off polyether sulfone membrane (Gelman), anodic separation compartment 40, pI=4.5 isoelectric separation barrier 50 (Gradipore) prepared from Immobiline™ chemicals (Amersham-Pharmacia), acrylamide and N-N′-methylene bis-acrylamide (Amersham-Pharmacia), cathodic separation compartment 60, and cathodic ion-permeable barrier 70 made of a 5,000 D nominal cut-off polyether sulfone membrane (Gelman). 80 mL of a 50 mM phosphoric acid solution (approximate pH=1.8) was circulated through anode compartment 15 at a flow rate of 2 L/min. 80 mL of a 50 mM sodium hydroxide solution (approximate pH=12.4) was circulated through cathode compartment 85 at a flow rate of 2 L/min. 38 mL of the liquid recovered from anodic separation compartment 40 after the IET separation in Example 3 using isoelectric separation barrier 50 with a pI value of 4.8 was circulated through anodic separation compartment 40 at a flow rate of 20 mL/min. Thus, initially, both the pI<4.8 proteins and the glutamic acid added in Example 3 were present in this stream. 40 mL of a 20 mM lysine solution was circulated through cathodic separation compartment 60 at a flow rate of 20 mL/min. Anode 10 was placed into anode compartment 15, and cathode 90 was placed into cathode compartment 85. The applied potential was 250 V, producing an initial current of 87 mA. After 6 passes of the prefractionated chicken egg white solution, the current leveled off at 68 mA. Aliquots were taken from anodic and cathodic separation compartments 40 and 60 after each pass and analyzed by the full-column UV absorbance imaging capillary IEF instrument as described in Example 1.

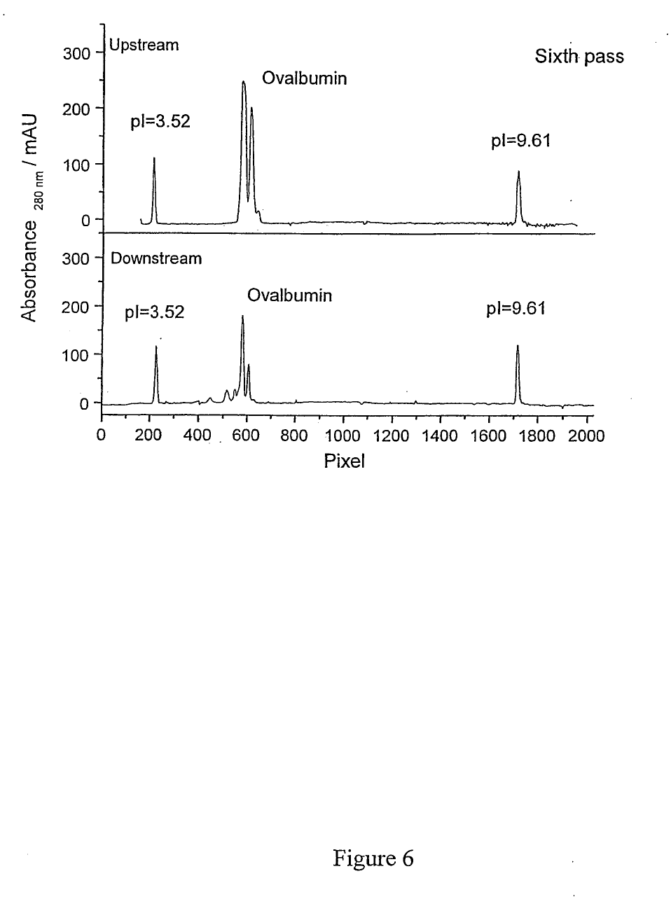

As a result of the sequentially applied second IET separation, proteins with pI values lower than 4.5 remained in anodic separation compartment 40 on the anodic side of pI=4.5 isoelectric separation barrier 50 (bottom panel in FIG. 6 labeled “Downstream”). Proteins with pI values greater than 4.5, such as ovalbumin (approximate pI=4.6) accumulated in cathodic separation compartment 60 on the cathodic side of isoelectric separation barrier 50 (top panel in FIG. 6 labeled “Upstream”). The transfer of ovalbumin was about 90% complete in 6 passes, aided by the presence of glutamic acid in anodic separation compartment 40 and lysine in cathodic separation compartment 60. Notably, cathodic separation compartment 60 did not contain any of pI<4.5 proteins; these remained in anodic separation compartment 40. Anodic separation compartment 40 contained some left-over pI>4.5 proteins. Once again, visual inspection of the interior of the IET cartridge at the end of the IET separation according to the present application revealed that there was no protein precipitation in either anodic separation compartment 40 or cathodic separation compartment 60.

### Example 5

Preparative-Scale Separation of a Chicken Egg White Sample that Contains Ampholytic Components by an IET Separation

The preparative-scale separation of a sample that contains ampholytic components was carried out in the presence of one isoelectric buffer according to the present application under the conditions described below.

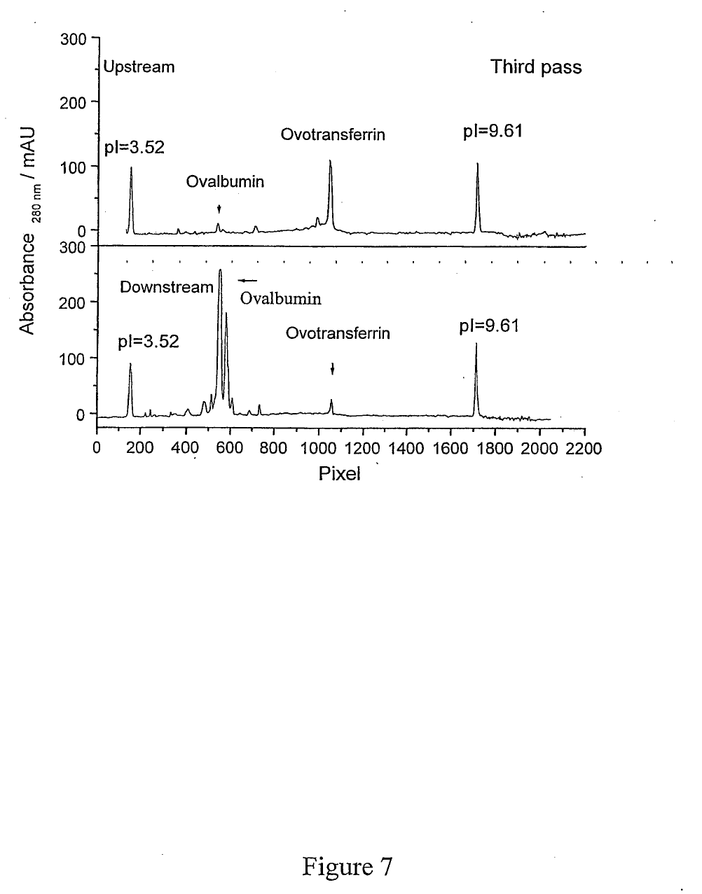

The separation according to the present application was obtained on the IET unit used in Examples 3 and 4 using an IET cartridge (U.S. Pat. No. 6,328,869) that contained anodic ion-permeable barrier 30 made of a 1,000 D nominal cut-off polyacrylamide membrane (Gradipore), anodic separation compartment 40, pI=5.0 isoelectric separation barrier 50 (Gradipore) prepared from Immobiline™ chemicals (Amersham-Pharmacia), acrylamide and N-N′-methylene bis-acrylamide (Amersham-Pharmacia), cathodic separation compartment 60, and cathodic ion-permeable barrier 70 made of a 1,000 D nominal cut-off polyacrylamide membrane (Gradipore). 60 mL of a 2 mM acetic acid solution (approximate pH=3.9) was circulated through anode compartment 15 at a flow rate of 2 L/min. 60 mL of a 15 mM ethanolamine solution (approximate pH=10.5) was circulated through cathode compartment 85 at a flow rate of 2 L/min. 30 mL of a filtered chicken egg-white solution (containing chicken egg white diluted at a rate of 1 to 25 in deionized water) was circulated through anodic separation compartment 40 at a flow rate of 20 mL/min. 30 mL of a filtered chicken egg-white solution (containing chicken egg white diluted at a rate of 1 to 25 in 5 mM lysine solution) was circulated through cathodic separation compartment 60 at a flow rate of 20 mL/min. Anode 10 was placed into anode compartment 15, and cathode 90 was placed into cathode compartment 85. The applied potential was 250 V that produced an initial current of 280 mA. Aliquots were taken from anodic and cathodic separation compartments 40 and 60 after each pass and analyzed by the full-column UV absorbance imaging capillary IEF instrument as described in Example 1. The IET separation was almost complete after 3 passes of the chicken egg white solutions, as shown in FIG. 7, requiring a total separation time of 6 minutes.

As a result of the IET separation, proteins with pI values lower than 5.0, such as ovalbumin (approximate pI=4.6), accumulated in anodic separation compartment 40 on the anodic side of pI=5.0 isoelectric separation barrier 50 (bottom panel in FIG. 7 labeled “Downstream”). Proteins with pI values greater than 5.0, such as ovotransferrin (approximate pI=6.1) accumulated in cathodic separation compartment 60 on the cathodic side of isoelectric separation barrier 50 (top panel in FIG. 7 labeled “Upstream”). Practically all of ovalbumin and the lower pIproteins transferred into anodic separation compartment 40.

### Example 6

Preparative-Scale Desalting of an Ampholytic Sample Mixture by an IET Separation

The preparative-scale desalting of an ampholytic component-containing sample was carried out in the presence of one isoelectric buffer according to the present application under the conditions described below.

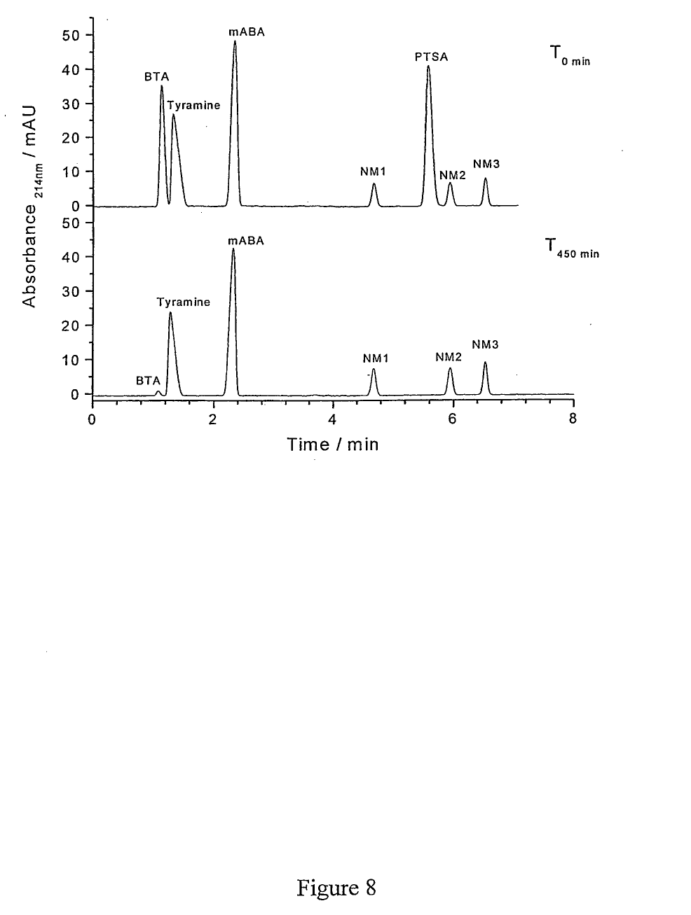

The IET desalting process according to the present application was obtained on the IET unit used in Example 2. The IET cartridge (U.S. Pat. No. 6,328,869) contained anodic ion-permeable barrier 30 made of a cross-linked poly(vinyl alcohol) membrane (Gradipore) and cathodic ion-permeable barrier 70 made of a cross-linked poly(vinyl alcohol) (Gradipore) membrane. 200 mL of an 80 mM phosphoric acid solution (approximate pH=1.6) was circulated through anode compartment 15 at a flow rate of 2 L/min. 200 mL of a 400 mM sodium hydroxide solution (approximate pH=13.2) was circulated through cathode compartment 85 at a flow rate of 2 L/min. 30 mL of sample solution (initially containing 30 mM tyramine as an ampholytic sample component, 30 mM m-aminobenzoic acid as an isoelectric buffer and 30 mM benzyltrimethylammonium para-toluene sulfonate as a strong electrolyte salt) was circulated through the separation compartment, at a flow rate of 20 mL/min. Anode 10 was placed into anode compartment 15, and cathode 90 was placed into cathode compartment 85. Initially, the applied potential was 5 V that produced a current of 500 mA. The recycled solution was electrophoresed for 450 minutes to demonstrate that ampholytic sample component tyramine could be trapped in the IET unit in a non-isoelectric state (mixed with isoelectric buffer m-aminobenzoic acid) for very long periods of time (7.5 hours). At 450 minutes, an applied potential of 80 V generated an electrophoretic current of 300 mA. Aliquots were taken from separation compartment over the course of the experiment and analyzed by pressure mediated capillary electrophoresis (PreMCE) as described in W. A. Williams and G. Vigh, “A Fast, Accurate Mobility Determination Method for Capillary Electrophoresis”, Analytical Chemistry 68 (1996) 1174. The PreMCE UV absorbance detector trace of the initial sample is shown in the top panel of FIG. 8 (labeled T_{0 min}), that of the last aliquot is shown in the bottom panel of FIG. 8 (labeled T_{450 min}). In the detector traces, label BTA indicates the peak corresponding to the benzyltrimethylammonium ion, label PTSA indicates the peak corresponding to the para-toluene sulfonate ion, label mABA indicates the peak corresponding to m-aminobenzoic acid, while labels NM1, NM2 and NM3 indicate the peaks corresponding to nitromethane, an internal standard in PreMCE analysis.

As a result of the IET separation according to the present application, strong electrolyte salt benzyltrimethylammonium para-toluene sulfonate was removed from the sample that contained ampholytic component tyramine. Tyramine was trapped in the separation compartment along with m-aminobenzoic acid and was in its non-isoelectric state. Thus, the initial composition of the sample has been successfully altered and the ampholytic sample component was trapped in the IET unit in its non-isoelectric state.

### Example 7

### Preparative-Scale Desalting of a Chicken Egg White Sample by an IET Separation

The preparative-scale desalting of a sample that contains ampholytic components was carried out in the presence of two isoelectric buffers according to the present application under the conditions described below.

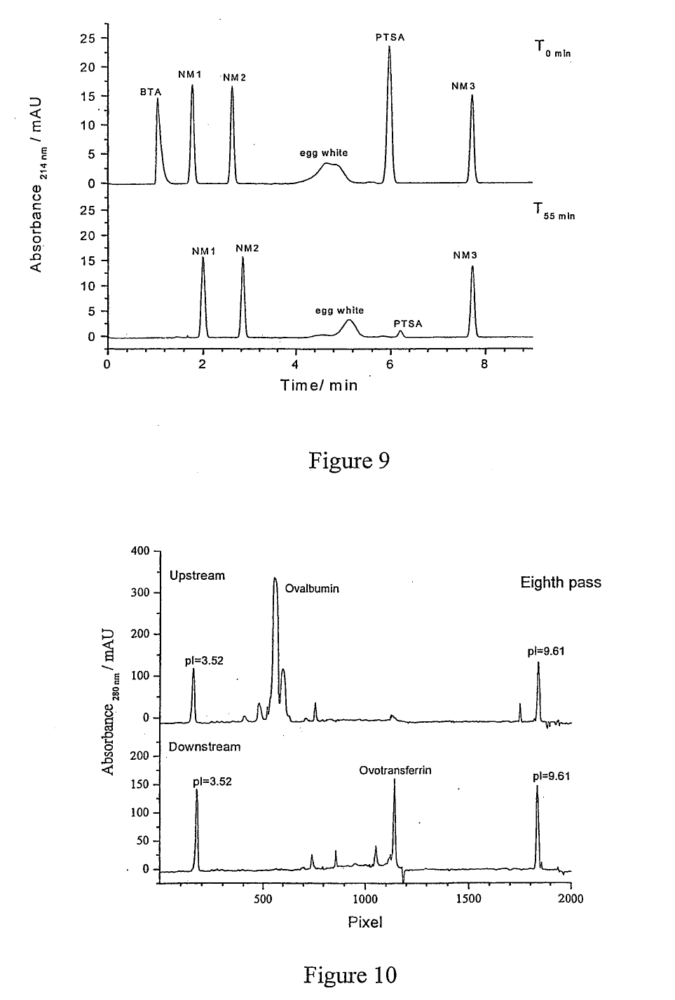

The IET desalting process according to the present application was carried out on the IET unit used in Example 2. The IET cartridge (U.S. Pat. No. 6,328,869) contained anodic ion-permeable barrier 30 made of a 5,000 D nominal cut-off polyether sulfone membrane (Gelman) and cathodic ion-permeable barrier 70 made of a 5,000 D nominal cut-off polyether sulfone membrane (Gelman). 200 mL of a 50 mM phosphoric acid solution (approximate pH=1.8) was circulated through anode compartment 15 at a flow rate of 2 L/min. 200 mL of a 50 mM sodium hydroxide solution (approximate pH=12.4) was circulated through cathode compartment 85 at a flow rate of 2 L/min. 80 mL of a filtered chicken egg white solution (containing chicken egg white as ampholytic sample components dissolved at a rate of 1 to 25 in a solution that also contained 20 mM glutamic acid as first isoelectric buffer, 20 mM lysine as second isoelectric buffer, and 25 mM benzyltrimethylammonium para-toluene sulfonate as strong electrolyte salt) was circulated through the separation compartment at a flow rate of 40 mL/min. Anode 10 was placed into anode compartment 15, and cathode 90 was placed into cathode compartment 85. The initially applied potential was 35 V that produced a current of 500 mA. The bulk of benzyltrimethylammonium para-toluene sulfonate salt was removed from the recirculated chicken egg white solution in as little as 55 minutes, as indicated by the finishing current leveling off at 24 mA. Aliquots were taken from the separation compartment over the course of the experiment and analyzed by pressure mediated capillary electrophoresis (PreMCE) as described in W. A. Williams and G. Vigh, “A Fast, Accurate Mobility Determination Method for Capillary Electrophoresis”, Analytical Chemistry 68 (1996) 1174. The PreMCE UV absorbance detector trace of the initial sample is shown in the top panel of FIG. 9 (labeled T_{0 min}), that of the last aliquot is shown in the bottom panel of FIG. 9 (labeled T_{55 min}) In the detector traces, label BTA indicates the peak corresponding to the benzyltrimethylammonium ion, label PTSA indicates the peak corresponding to the para-toluene sulfonate ion, while labels NM1, NM2 and NM3 indicate the peaks corresponding to nitromethane, an internal standard in PreMCE analysis.

As a result of the IET separation according to the present application, the strong electrolyte salt, benzyltrimethylammonium para-toluene sulfonate was removed from the sample, while the ampholytic sample components, the chicken egg white proteins were trapped in the separation compartment in their non-isoelectric state, mixed both with lysine and glutamic acid. Thus, the initial composition of the sample has been successfully altered and the ampholytic sample components were trapped in the IET unit in their non-isoelectric state in the presence of two isoelectric buffers.

### Example 8

Preparative-Scale Separation of a Chicken Egg White Sample by an IET Separation

The preparative-scale separation of a sample that contains ampholytic components was carried out in the presence of two isoelectric buffers according to the present application under the conditions described below.

The IET separation according to the present application was obtained on the IET unit used in Example 2. The IET cartridge (U.S. Pat. No. 6,328,869) contained anodic ion-permeable barrier 30 made of a pI=3.0 isoelectric barrier (Gradipore) prepared from Immobiline® chemicals (Amersham-Pharmacia), acrylamide and N-N′-methylene bis-acrylamide (Amersham-Pharmacia), anodic separation compartment 40, pI=5.0 isbelectric separation barrier 50 (Gradipore) prepared from Immobiline™ chemicals (Amersham-Pharmacia), acrylamide and N-N′-methylene bis-acrylamide (Amersham-Pharmacia), cathodic separation compartment 60, and cathodic ion-permeable barrier 70 made of a pI=8.2 isoelectric barrier (Gradipore) prepared from Immobiline™ chemicals (Amersham-Pharmacia), acrylamide and N-N′-methylene bis-acrylamide (Amersham-Pharmacia). 80 mL of a 10 mM phosphoric acid solution was circulated through anode compartment 15 at a flow rate of 2 L/min. 80 mL of a 10 mM triethanolamine solution was circulated through cathode compartment 85 at a flow rate of 2 L/min. 30 mL each of a filtered chicken egg white solution (containing chicken egg white dissolved in 20 mM glutamic acid and 20 mM histidine, respectively, at a dilution rate of 1 to 25) was circulated through anodic and cathodic separation compartments 40 and 60, respectively, at a flow rate of 20 mL/min. Anode 10 was placed into anode compartment 15, and cathode 90 was placed into cathode compartment 85. The initial electrophoresis current was set at 250 mA. The separation was practically complete after 8 passes of the chicken egg white solution through the separation compartments, resulting in a total separation time of 16 minutes. Aliquots were taken from anodic and cathodic separation compartments 40 and 60 after each pass and analyzed by the full-column UV absorbance imaging capillary IEF instrument as described in Example 1.

As a result of the IET separation according to the present application, proteins with pI values lower than 5.0, such as ovalbumin (approximate pI 4.6), accumulated in anodic separation compartment 40 on the anodic side of pI=5.0 isoelectric separation barrier 50 (bottom panel in FIG. 10 labelled “Downstream”). Proteins with pI values greater than 5.0, such as ovotransferrin (approximate pI=6.1) accumulated in cathodic separation compartment 60 on the cathodic side of isoelectric separation barrier 50 (top panel in FIG. 10 labelled “Upstream”). Practically all of ovalbumin and the lower pI proteins transferred into anodic separation compartment 40. Visual inspection of the interior of the separation cartridge at the end of this IET separation indicated that protein precipitation did not occur either in anodic separation compartment 40 or cathodic separation compartment 60.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for separating an ampholytic component by electrophoresis, the method comprising:
placing a sample containing an ampholytic component having a pI value in an electrophoresis separation system comprising an anolyte having a pH and a catholyte having a pH, the catholyte pH being higher than the anolyte pH, one or more ion-permeable barriers disposed between the anolyte and catholyte wherein at least one of the barriers is an isoelectric barrier having a pI value which is higher than the anolyte pH and lower than the catholyte pH;
providing an isoelectric buffer having a pI value higher than the pH of the anolyte and lower than the pH of the catholyte and different from the pI value of the ampholytic sample component and different from the pI value of an ion-permeable isoelectric barrier; and
exposing the sample to an electric potential so as to trap the ampholytic sample component in a non-isoelectric state in the presence of the isoelectric buffer in the electrophoresis system.

2. The method according to claim 1 wherein the electrophoretic separation system comprises an anode compartment containing an anode and adapted to receive the anolyte, a cathode compartment containing a cathode and adapted to receive the catholyte, and one ion-permeable barrier in the form of an ion-permeable isoelectric barrier having a pI value disposed between the anode compartment and the cathode compartment.

3. The method according to claim 2 wherein the electrophoretic separation system further comprises first and second ion-permeable barriers disposed between the anode compartment and the cathode compartment forming a first separation compartment between the anode compartment and cathode compartment.

4. The method according to claim 3 wherein the first and second ion-permeable barriers are ion-permeable isoelectric barriers each having a defined but different pI.

5. The method according to claim 3 wherein the sample is provided to at least one of the sample compartment, anode compartment or the cathode compartment.

6. The method according to claim 5 wherein the sample is provided to the sample compartment and the ampholytic component is separated in a non-isoelectric state in the sample compartment.

7. The method according to claim 5 wherein the ampholytic component is obtained in a non-isoelectric state in the sample compartment or the cathode compartment or the anode compartment.

8. The method according to claim 3 wherein the sample contains two or more ampholytic components which are obtained in non-isoelectric states in the sample compartment or the cathode compartment or the anode compartment or two or more of the sample compartment, the anode compartment or the cathode compartment.

9. The method according to claim 3 wherein the electrophoretic separation system further comprises another ion-permeable barrier disposed between the anode compartment and the cathode compartment forming a second separation compartment adjacent the first separation compartment, the system having first ion-permeable isoelectric barrier having a first pI value and a second ion-permeable isoelectric barrier having a second pI value different from the first pI value of the first ion-permeable isoelectric barrier.

10. The method according to claim 9 wherein the ion-permeable barriers are all isoelectric barriers each having a defined but different pI.

11. The method according to claim 9 wherein the sample is provided to the first sample compartment, the second sample compartment, the anode compartment or the cathode compartment, or both the first and second sample compartments.

12. The method according to claim 11 wherein the ampholytic component is obtained in a non-isoelectric state in the first sample compartment or in the second sample compartment, or in the anode compartment, or the cathode compartment.

13. The method according to claim 9 wherein the sample contains two or more ampholytic components which are obtained in a non-isoelectric state in the first sample compartment or in the second sample compartment, or in each of the first sample compartment and second sample compartment.

14. The method according to claim 2 wherein the electrophoretic separation system further comprises a plurality of ion-permeable barriers disposed between the anode compartment and the cathode compartment forming a plurality of separation compartments disposed between the anode and cathode compartments, wherein two or more ion-permeable barriers are ion-permeable isoelectric barriers each having a different pI value.

15. The method according to claim 14 wherein three or more of the ion-permeable barriers are ion-permeable isoelectric barriers each having a different pI value.

16. The method according to claim 14 wherein all the ion-permeable barriers are ion-permeable isoelectric barriers each having a different pI value.

17. The method according to claim 9 wherein a first isoelectric buffer and a second isoelectric buffer each having a different pI value are provided to at least two of the sample compartments or the anode compartment or the cathode compartment.

18. The method according to claim 1 wherein the ion-permeable barriers are selected from the group consisting of immiscible liquids, porous solids, non-ionic membranes, membranes, isoelectric membranes, hydrogel membranes, gels, and hydrogels.

19. The method according to 18 wherein the hydrogel membranes are polyether sulfone-based, poly(vinyl) alcohol-based or polyacrylamide-based.

20. The method according to claim 18 wherein the non-ionic membranes are supported membranes selected from the group consisting of cross-linked polyacrylamide and poly(vinyl) alcohol supported on glass fiber, filter paper, polymeric mesh, and paper.

21. The method according to claim 18 wherein the ion-permeable barriers are porous frits selected from the group consisting of glass frits, ceramic frits, and polymeric frits.

22. The method according to claim 18 wherein the ion-permeable barriers substantially restrict the passage of molecules having a size or hydrated ion radius greater than a predetermined size.

23. The method according to claim 1 wherein the ion-permeable isoelectric barriers are selected from the group consisting of isoelectric porous solids, isoelectric membranes, and isoelectric gels.

24. The method according to claim 23 wherein the ion-permeable isoelectric barriers are isoelectric membranes.

25. The method according to claim 1 wherein there is substantially no convective mixing between the compartments.

26. The method according to claim 1 wherein the isoelectric buffer is provided to at least one of a sample compartment, the anode compartment and the cathode compartment.

27. The method according to claim 26 wherein the pI value of the isoelectric buffer differs by at least 0.001 pH units from the pI value of the ampholytic sample component.

28. The method according to claim 1 wherein the absolute value of the difference between the pI value and the pKa value closest to the pI value of the isoelectric buffer is less than about 1.5.

29. The method according to claim 1 wherein the isoelectric buffer is selected from the group consisting of iminodiacetic acid, N-methylimino diacetic acid, aspartic acid, glutamic acid, glycyl-aspartic acid, m-aminobenzoic acid, histidyl-glycine, histidyl-histidine, histidine, 1,2-diaminopropionic acid, ornithine, lysine, lysil-lysine, and arginine.

30. The method according to claim 29 wherein the isoelectric buffer is glutamic acid or lysine.

31. The method according claim 17, wherein the first isoelectric buffer is glutamic acid and the second isoelectric buffer is lysine.

32. The method according to claim 1 further comprising providing a non-ionic solubilizing agent.

33. The method according to claim 32 wherein the solubilizing agent is a non-ionic detergent.

34. The method according to claim 1 wherein the ampholytic sample component is selected from the group consisting of proteins, polypeptides, oligopeptides, amino phenols, amino phosphonic acids, and amino acids.

35. The method according to claim 34, wherein the ampholytic sample component is a protein.
